(19) United States
(12) Patent Application Publication
(10) Pub. No.: US 2024/0397917 A1
(43) Pub. Date: Dec. 5, 2024

(54) ANIMAL PRODUCTION METHOD

(71) Applicant: DSM IP ASSETS B.V., Heerlen (NL)

(72) Inventors: Marco CESCO, Kaiseraugst (CH); Carlos LOZANO, Kaiseraugst (CH); Claudia SILVA, Kaiseraugst (CH)

(21) Appl. No.: 18/699,673

(22) PCT Filed: Oct. 12, 2022
(86) PCT No.: PCT/EP2022/078402

§ 371 Date: Apr. 9, 2024

(30) Foreign Application Priority Data
Oct. 12, 2021 (EP) 21202130.7

(51) Int. Cl. A01K 67/02; G01N 33/68; G01N 33/82; G01N 33/84

(52) U.S. Cl. CPC A01K 67/02; G01N 33/6848; G01N 33/82; G01N 33/84

(57) ABSTRACT

The present invention relates to industrial animal production. In particular the present invention relates to a method of raising a group of animals, and a method of determining analytes, in egg yolk or in the blood of an animal by ex vivo analysis of blood and/or yolk samples.

### TECHNICAL FIELD

The present invention relates to industrial animal production. In particular the present invention relates to a method of raising a group of animals and a method of determining analytes in the blood and/or egg yolk of an animal.

### BACKGROUND OF THE INVENTION

Animals are raised for various reasons, including egg and meat production. At industrial scale, a farmer is responsible for hundreds or thousands of animals: the larger the number of animals, the more efficient mass production is. Managing animal health, welfare and performance is of utmost importance in industrial animal production.

One approach to increase profitability of industrial animal production is precision animal nutrition. To optimize profitability, the farmer attempts to influence animal's performance by adapting the nutrition to the specific needs of his animals.

In order to benefit from precision animal nutrition, there is a need to measure accurately the analyte status and relate the status to animal health and performance. It is commonly known that analytes in the feed do not always correlate with the bioavailability and blood levels of said analytes. What really matters is how much analyte it is bioavailable for the animal. Therefore, there is a need for a practical tool that easily allows to measure the analyte status of an animal, at any time.

### SUMMARY OF THE INVENTION

The present invention relates to industrial animal production. In particular the present invention relates to a method of raising a group of animals and a method of determining analytes in the blood and/or egg yolk of an animal.

The present invention relates to a method of determining at least one analyte in the blood of an animal, said method comprising the steps:
i) providing a drop of blood from an animal,
ii) collecting the blood provided in step i) on a carrier,
iii) letting the blood on the carrier dry, and
iv) analyzing the blood on the carrier provided in step iii) to measure the values of at least one analyte parameter.

The method may further comprise the steps of
v) Comparing the values of the at least one analyte measured in step iv) with the levels of the at least one analyte recommended in nutritional guidelines, and
vi) adapting feed that is fed to the animal, if the values of the at least one analyte measured in step iv) deviate from ranges recommended in the nutritional guidelines, to ensure recommended animal nutrient levels according to the nutritional guidelines.

In a preferred embodiment, the blood is venous blood.

In a preferred embodiment, the animals are swine or poultry, preferably broiler chicken.

In a preferred embodiment, the analysis in step iv) is performed by liquid chromatography coupled with mass spectrometry (LC-MS/MS).

The method can be improved by simultaneous measurements of more than one analyte. In a preferred embodiment, the values of at least two, preferably at least three, more preferably of at least four analytes are measured in step iv).

In another embodiment, the present invention relates to a method of determining at least one analyte in egg yolk, said method comprising the steps:
i) providing a drop of egg yolk,
ii) collecting the egg yolk provided in step i) on a carrier,
iii) letting the egg yolk on the carrier dry, and
iv) analyzing the egg yolk on the carrier provided in step ii) to measure the values of at least one analyte in egg yolk.

The method may further comprise the steps of
v) Comparing the values of the at least one analyte measured in step iv) with the levels of the at least one analyte recommended in nutritional guidelines, and
vi) adapting feed that is fed to the animal, if the values of the at least one analyte measured in step iv) deviate from ranges recommended in the nutritional guidelines, to ensure recommended animal nutrient levels according to the nutritional guidelines.

In a preferred embodiment, the animals are swine or poultry, preferably broiler chicken.

In a preferred embodiment, the analysis in step iv) is performed by liquid chromatography coupled with mass spectrometry (LC-MS/MS).

The method can be improved by simultaneous measurements of more than one analyte. In a preferred embodiment, the values of at least two, preferably at least three, more preferably of at least four analytes are measured in step iv).

The present invention further relates to a method of raising a group of animals of same species and same breed, said method comprising the steps:
i) providing a drop of blood from at least one member of the group,
ii) collecting the blood provided in step i) on a carrier,
iii) letting the blood on the carrier dry,
iv) analyzing the blood on the carrier provided in step iii) to measure the values of at least one analyte, and
v) Comparing the values of the at least one analyte measured in step iv) with the levels of the at least one analyte recommended in nutritional guidelines
vi) adapting feed that is fed to the animal, if the values of the at least one analyte measured in step iv) deviate from ranges recommended in the nutritional guidelines, to ensure recommended animal nutrient levels according to the nutritional guidelines.

In a preferred embodiment, the blood is venous blood.

In a preferred embodiment, the animals are swine or poultry, preferably broiler chicken.

In a preferred embodiment, the analysis in step iv) is performed by liquid chromatography coupled with mass spectrometry (LC-MS/MS).

The method of the invention allows for measuring the analyte status of an animal from which the blood sample has been taken and subsequent adaptation of the feed that is fed to the animal.

The method can be improved by simultaneous measurements of more than one analyte. In a preferred embodiment, the values of at least two, preferably at least three, more preferably of at least four analytes are measured in step iv).

In another embodiment, the present invention relates to a method of raising a group of animals of same species and same breed, said method comprising the steps:
i) providing a drop of egg yolk from an egg of least one member of the group,
ii) collecting the egg yolk provided in step i) on a carrier,
iii) letting the egg yolk on the carrier dry,
iv) analyzing the egg yolk on the carrier provided in step iii) to measure the values of at least one analyte, and
v) Comparing the values of the at least one analyte measured in step iv) with the levels of the at least one analyte recommended in nutritional guidelines
vi) adapting feed that is fed to the animal, if the values of the at least one analyte measured in step iv) deviate from ranges recommended in the nutritional guidelines, to ensure recommended animal nutrient levels according to the nutritional guidelines.

In a preferred embodiment, the animals are swine or poultry, preferably broiler chicken.

In a preferred embodiment, the analysis in step iv) is performed by liquid chromatography coupled with mass spectrometry (LC-MS/MS).

The method of the invention allows for measuring the analyte status of an animal from which the blood sample has been taken and subsequent adaptation of the feed that is fed to the animal.

The method can be improved by simultaneous measurements of more than one analyte. In a preferred embodiment, the values of at least two, preferably at least three, more preferably of at least four analytes are measured in step iv).

In a preferred embodiment, the animals are swine or poultry, preferably broiler chicken.

Due to the accuracy of the measurement, adequate measures can be taken to mitigate potential loss. Such measures include adaption of the animals' nutrition.

In preferred embodiment, the feed in step vi) of the method of the invention is adapted by adjusting the amount of at least one feed additive.

### DETAILED DESCRIPTION OF THE INVENTION

### Method of Determining an Analyte Parameter in the Blood of an Animal

A preferred embodiment of the method of the invention relates to a method of determining a analyte in the blood of an animal.

In a preferred embodiment of the invention, the animals are birds such as chicken or ducks. Even more preferably, the animals are broilers. Well known breeds are Ross (e.g. Ross 708) and Cobb (e.g. Cobb 500).

In another preferred embodiment of the invention, the animals are swine.

In step iv) of the method of the invention, a blood sample of an animal is analysed ex vivo. The blood sample analysed in step iv) comprises preferably venous blood. Thus, preceding step i) of the method of the invention comprises preferably the provision of venous blood an animal, wherein the method of the present invention is preferably not a diagnostic method practised on the animal body.

In step iv) of the method of the invention, the venous blood provided in step i), collected on a carrier in step ii) and dried in step iii) is analyzed ex vivo. The blood sample is preferably taken at the place where the animal is raised. The method of the invention comprises preferably means to draw venous blood from an animal. An example of the sampling procedure is provided in example 2.

The sample may be send directly to the Laboratory using a carrier card which eliminates the biological risk. Compared to traditional blood samples, that are difficult to handle and need refrigeration, that cannot be sent across borders or by mail, for the method of the present invention no refrigeration is required for transportation and subsequent storage. It is safe for sending all over the world, without biological risks, or requiring imports permits.

The analysis is preferably done in a laboratory to which the sample has been mailed. Analysis of the sample on the carrier is performed through conventional methods, such as, for example mass analysis by liquid chromatography (HPLC) or High Efficiency Liquid Chromatography Coupled with Mass Spectrometry (LC-MS/MS) to determine the level of analyte in the animal. This procedure is highly correlated with the conventional HPLC test of blood plasma for said analyte.

Preferably, the decision which blood parameters will be analysed in step vi) has been taken before doing the analysis in step iv).

The optional correlation performed in step v), may correlate analyte the levels of said analyte to improve health and performance factors.

The optional adaptation of the feed performed in step vi) to the values of the analyte may be facilitated by ordering feed or a feed additive from an external supplier.

The procedure of the method of the present invention comprised in steps i) to iv), for the purpose of this invention will be summarized as dried Blood Spot Test (DBS). DBS allows for sampling the animals in an easier and less invasive way, since only one drop of the animal's blood is needed. Considering the issues of sending test materials, the DBS is a safer and less complicated technique because the carrier cards preserve the material for longer periods of time, inhibiting the presence of any infectious agent and allowing samples to be easily transported abroad or stored at room temperature. All this helps to improve the quality and agility in sending the samples.

### Method of Determining an Analyte Parameter in Egg Yolk

A preferred embodiment of the method of the invention relates to a method of determining a analyte in egg yolk.

In a preferred embodiment of the invention, the animals are birds such as chicken or ducks. Even more preferably, the animals are broilers. Well known breeds are Ross (e.g. Ross 708) and Cobb (e.g. Cobb 500).

In step iv) of the method of the invention, the yolk provided in step i), collected on a carrier in step ii) and dried in step iii) is analyzed ex vivo. The yolk sample is preferably taken at the place where the animal is raised. An example of the sampling procedure is provided in example 3. The sample may be send directly to the Laboratory using a carrier card which eliminates the biological risk. Compared to traditional biological samples, that are difficult to handle and need refrigeration, that cannot be sent across borders or by mail, for the method of the present invention no refrigeration is required for transportation and subsequent storage. It is safe for sending all over the world, without biological risks, or requiring imports permits.

The analysis is preferably done in a laboratory to which the sample has been mailed. Analysis of the sample on the carrier is performed through conventional methods, such as, for example mass analysis by liquid chromatography (HPLC) or High Efficiency Liquid Chromatography Coupled with Mass Spectrometry (LC-MS/MS) to determine the level of analyte in the yolk.

Preferably, the decision which blood parameters will be analysed in step vi) has been taken before doing the analysis in step iv).

The optional correlation performed in step v), may correlate analyte the levels of said analyte to improve health and performance factors.

The optional adaptation of the feed performed in step vi) to the values of the analyte may be facilitated by ordering feed or a feed additive from an external supplier.

The procedure of the method of the present invention comprised in steps i) to iv), for the purpose of this invention will be summarized as Dried Yolk Spot Test (DYS). DYS allows for sampling the animals in an easier and less invasive way, since only one drop of yolk is needed. Considering the issues of sending test materials, the DYS is a safer and less complicated technique because the carrier cards preserve the material for longer periods of time, inhibiting the presence of any infectious agent and allowing samples to be easily transported abroad or stored at room temperature. All this helps to improve the quality and agility in sending the samples.

### Method of Raising a Group of Animals, Wherein the Blood of an Animal is Analyzed

A preferred embodiment of the method of the invention relates to a method of a raising a group of animals of same species, same breed.

The number of animals in the group of animals can vary and depends on the species. In a preferred embodiment of the invention, the animals are birds such as chicken or ducks. Even more preferably, the animals are broilers. Well known breeds are Ross (e.g. Ross 708) and Cobb (e.g. Cobb 500). Thus, a preferred embodiment of the method of the invention relates to a method of raising a group of birds, preferably chicken, of same species and same breed.

In another preferred embodiment of the invention, the animals are swine.

Preferably, the group of animals are raised in the same compartment, e.g. in the same pen or in the same house. In case the animals are chicken, a pen typically comprises up to 100 chicken whereas a chicken house may comprise 1,000 or more chicken. Thus, one embodiment of the invention relates to a method of raising a group of animals of same species and same breed, wherein said group is raised in the same compartment and/or wherein said group comprises more than 10, preferably more than 50, even more preferably more than 100 and most preferably at least 1,000 animals.

In step iv) of the method of the invention, a blood sample of at least one member of the group of animals is analysed ex vivo. Whereas it is possible to analyse blood samples of more than one member of the group, it is typically sufficient to analyse a blood sample of only one member of the group. Thereby, the at least one member of the group is preferably selected randomly from the group of animals. The blood sample analysed in step iv) comprises preferably venous blood. Thus, preceding step i) of the method of the invention comprises preferably the provision of venous blood from at least one member of the group, wherein the method of the present invention is preferably not a diagnostic method practised on the animal body

In step iv) of the method of the invention, the venous blood provided in step i), collected on a carrier in step ii) and dried in step iii) is analyzed ex vivo. The blood sample is preferably taken at the place where the animals are raised. The method of the invention comprises preferably means to draw venous blood from an animal. An example of the sampling procedure is provided in example 2. The sample may be send directly to the Laboratory using a carrier card which eliminates the biological risk. Compared to traditional blood samples, that are difficult to handle and need refrigeration, that cannot be sent across borders or by mail, for the method of the present invention no refrigeration is required for transportation and subsequent storage. It is safe for sending all over the world, without biological risks, or requiring imports permits.

The analysis is preferably done in a laboratory to which the sample has been mailed. Analysis of the sample on the carrier is performed through conventional methods, such as, for example mass analysis by liquid chromatography (HPLC) or High Efficiency Liquid Chromatography Coupled with Mass Spectrometry (LC-MS/MS) to determine the level of analyte in the animal. This procedure is highly correlated with the conventional HPLC test of blood plasma for said analyte.

Preferably, the decision which blood parameters will be analysed in step vi) has been taken before doing the analysis in step iv).

The correlation performed in step v), may correlate analyte the levels of said analyte to improve health and performance factors.

The adaptation of the feed performed in step vi) to the values of the analyte may be facilitated by ordering feed or a feed additive from an external supplier. Thus, a preferred embodiment of the invention relates to a set-up comprising the herein described method of raising a group of animals and at least one supplier of feed additives, premixes and/or feed, wherein the results of the analysis step iv) or the correlation of step v) is communicated to the at least one supplier of feed additives, premixes and/or feed.

The procedure of the method of the present invention comprised in steps i) to iv), for the purpose of this invention will be summarized as dried Blood Spot Test (DBS). DBS allows for sampling the animals in an easier and less invasive way, since only one drop of the animal's blood is needed. Considering the issues of sending test materials, the DBS is a safer and less complicated technique because the carrier cards preserve the material for longer periods of time, inhibiting the presence of any infectious agent and allowing samples to be easily transported abroad or stored at room temperature. All this helps to improve the quality and agility in sending the samples.

### Method of Raising a Group of Animals, Wherein Egg Yolk is Analyzed

A preferred embodiment of the method of the invention relates to a method of a raising a group of animals of same species, same breed.

The number of animals in the group of animals can vary and depends on the species. In a preferred embodiment of the invention, the animals are birds such as chicken or ducks. Even more preferably, the animals are broilers. Well known breeds are Ross (e.g. Ross 708) and Cobb (e.g. Cobb 500). Thus, a preferred embodiment of the method of the invention relates to a method of raising a group of birds, preferably chicken, of same species and same breed.

Preferably, the group of animals are raised in the same compartment, e.g. in the same pen or in the same house. In case the animals are chicken, a pen typically comprises up to 100 chicken whereas a chicken house may comprise 1,000 or more chicken. Thus, one embodiment of the invention relates to a method of raising a group of animals of same species and same breed, wherein said group is raised in the same compartment and/or wherein said group comprises more than 10, preferably more than 50, even more preferably more than 100 and most preferably at least 1,000 animals.

In step iv) of the method of the invention, a yolk sample of an egg of at least one member of the group of animals is analysed. Whereas it is possible to analyse yolk samples of eggs of more than one member of the group, it is typically sufficient to analyse a yolk sample of only one member of the group. Thereby, the at least one member of the group is preferably selected randomly from the group of animals.

The yolk sample is preferably taken at the place where the animals are raised. The method of the invention comprises preferably means to draw a sample from an egg. An example of the sampling procedure is provided in example 3. The sample may be send directly to the Laboratory using a carrier card which eliminates the biological risk. Compared to traditional biological samples, that are difficult to handle and need refrigeration, that cannot be sent across borders or by mail, for the method of the present invention no refrigeration is required for transportation and subsequent storage. It is safe for sending all over the world, without biological risks, or requiring imports permits.

The analysis is preferably done in a laboratory to which the sample has been mailed. Analysis of the sample on the carrier is performed through conventional methods, such as, for example mass analysis by liquid chromatography (HPLC) or High Efficiency Liquid Chromatography Coupled with Mass Spectrometry (LC-MS/MS) to determine the level of analyte in the animal.

Preferably, the decision which yolk parameters will be analysed in step vi) has been taken before doing the analysis in step iv).

The correlation performed in step v, may correlate analyte the levels of said analyte to improve health and performance factors.

The adaptation of the feed performed in step vi) to the values of the analyte may be facilitated by ordering feed or a feed additive from an external supplier.

Thus, a preferred embodiment of the invention relates to a set-up comprising the herein described method of raising a group of animals and at least one supplier of feed additives, premixes and/or feed, wherein the results of the analysis step iv) or the correlation of step v) is communicated to the at least one supplier of feed additives, premixes and/or feed.

The procedure of the method of the present invention comprised in steps i) to iv), for the purpose of this invention will be summarized as Dried Yolk Spot Test (DYS). DYS allows for sampling the animals in an easier and less invasive way, since only one drop of the animal's blood is needed. Considering the issues of sending test materials, the DYS is a safer and less complicated technique because the carrier cards preserve the material for longer periods of time, inhibiting the presence of any infectious agent and allowing samples to be easily transported abroad or stored at room temperature. All this helps to improve the quality and agility in sending the samples.

### Definitions

Analyte: In the context of the present invention, the term “analyte” refers to a component or chemical species that is of interest in an analytical procedure. In a preferred embodiment of the invention, the analyte may be a nutrient or a biomarker.

Animal: In the context of the present invention, the term “animal” includes all non-human members of the kingdom Animalia. Preferably, the animal is a mono-gastric animal, such as swine (including, but not limited to, piglets, growing pigs, and sows), poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

Preferably, the animal is selected from the group of pigs or swine (including, but not limited to, piglets, growing pigs, and sows) or poultry (including but not limited to poultry, turkey, duck, quail, guinea fowl, goose, pigeon, squab, chicken, broiler, layer, pullet and chick).

Anticoagulant: Sometimes, an anticoagulant is added to a blood sample. Well-known anticoagulants are heparin and EDTA. In the context of the present invention, blood containing heparin is referred to a “heparinized blood”.

Biomarker: Biomarkers (short for biological marker), for the purpose of this invention, are objective, quantifiable characteristics of biological processes. A biomarker is an objective measure that captures what is happening in a cell or an organism at a given moment. Biomarkers, for example, may help us understand relationships between environmental factors and diseases to improve our ability to diagnose, monitor, or predict disease risk. Biomarkers for the purpose of the present invention may be selected from, but not limited to Albumin, Calcium, Carotenoids, Creatine Kinease, Globulin, Glucose, Hemoglobin, Phosphorus, Potassium, Sodium, Total Carbon Dioxide, Uric Acid.

Albumin: In the context of the present invention, the term “Albumin” refers to a family of globular proteins. All the proteins of the albumin family are water-soluble, moderately soluble in concentrated salt solutions, and experience heat denaturation. Albumins are commonly found in blood plasma and differ from other blood proteins in that they are not glycosylated. Substances containing albumins are called albuminoids. The cconcentration of albumin in the blood serum (serum albumin, SA) may be influenced by several factors, including its synthesis rate, catabolismrate, extravascular distribution, and exogenous loss. Moreover, both nutritional status and systemic inflammation affect the synthesis of SA. Determining SA concentration aids in risk prediction in various clinical settings. An increased risk in all-cause mortality and cardiovascular (CV) mortality may be associated with low SA concentration.

Calcium: Calcium deficiency may lead to metabolic alterations or potential pathological changes. Therefore, calcium may serve as a biomarker to metabolic problems and/or pathological changes.

Carotenoids: In the context of the present invention, the term “Carotenoids” refers to organic pigments that are produced by plants and algae, as well as several bacteria, and fungi. Carotenoids are obtained from the diet as brightly coloured pigments. They may serve as biomarkers of fruit and vegetable intake, but not be limited to those.

Creatine Kinease: In the context of the present invention, the term “Creatine kinase” (CK) refers to an enzyme present in tissue and in energy-demanding cells, such as skeletal and cardiac muscles, and is considered the best marker for the detection and monitoring of skeletal muscle diseases Creatine kinase activity is typically measured in serum or plasma.

Globulin: In the context of the present invention, the term “globulin” refers to a family of globular proteins that have higher molecular weights than albumins and are insoluble in pure water but dissolve in dilute salt solutions. Some globulins are produced in the liver, while others are made by the immune system. Globulins, albumins, and fibrinogen are the major blood proteins. Higher globulin levels are strongly associated with the risk of Periprosthetic joint infection (PJI) and may serve as biomarkers in the diagnosis of PJI. The albumin/globulin ratio in blood serum is further recognized as a valuable prognostic biomarker in various cancers.

Glucose: In the context of the present invention, the term “Glucose” refers to a simple sugar with the molecular formula C₆H₁₂O₆. Glucose is the most abundant monosaccharide, a subcategory of carbohydrates. Glucose is mainly made by plants and most algae during photosynthesis from water and carbon dioxide, using energy from sunlight, where it is used to make cellulose in cell walls, the most abundant carbohydrate in the world. Continuous glucose monitoring provides detailed real-time data that is of value in clinical decision making, assessing response to new diabetes drugs and the development of closed-loop artificial pancreas technology.

Hemoglobin: In the context of the present invention, the term “Hemoglobin”, or haemoglobin, abbreviated Hb or Hgb, refersto the iron-containing oxygen-transport metalloprotein in the red blood cells (erythrocytes) of almost all vertebrates as well as the tissues of some invertebrates. Hemoglobin in blood carries oxygen from the lungs or gills to the rest of the body (i.e. the tissues). There it releases the oxygen to permit aerobic respiration to provide energy to power the functions of the organism in the process called metabolism. High hemoglobin level (Hb) may be associated with increased risk of total cancer and breast cancer incidence.

Phosphorus: In the context of the present invention, the term “Phosphorus” refers to a chemical element with the symbol P and atomic number 15. Elemental phosphorus exists in two major forms, white phosphorus and red phosphorus, but because it is highly reactive, phosphorus is never found as a free element on Earth. In minerals, phosphorus generally occurs as phosphate. Twenty-four-hour urine phosphorus is commonly used as a surrogate measure for phosphorus intake and absorption.

Potassium: In the context of the present invention, the term “Potassium” refers to a chemical element with the symbol K and atomic number 19. Potassium in nature occurs only in ionic salts. Potassium is the most abundant intracellular cation that plays a critical role in transmitting nerve impulse, cardiac activity, membrane transport, acid-base balance, and neuromuscular functions. Potassium-rich diets such as DASH [Dietary Approaches to Stop Hypertension] may lower blood pressure and decreased kidney disease progression.

Sodium: In the context of the present invention, the term “Sodium” refers to a chemical element with the symbol Na and atomic number 11. The free metal does not occur in nature. Excess sodium intake increases the risk for high blood pressure, and high blood pressure, or hypertension, is a leading risk factor for cardiovascular disease.

Total Carbon Dioxide: Carbon Dioxide is produced in the body from cellular respiration. As a biomarker, carbon dioxide in serum is really a measure of bicarbonate in the blood. The majority of CO₂ (about 75%), produced from cellular respiration, is carried in the blood as the bicarbonate ion. 5% remains in solution as dissolved CO₂ and the remaining 20% remains combined with hemoglobin and other plasma proteins. Dissolved CO₂, formed in the lungs, contributes little to the CO₂ value. In blood test, CO₂ or bicarbonate is as a general measure of tissue acidity or alkalinity. CO₂ content refers to bicarbonate, which is an alkaline or base molecule. It is a solution and is regulated primarily by the kidneys, referred to CO₂ on blood chemistry panels. CO₂ gas, on the other hand, refers to the dissolved CO₂ and is mainly acid. It is regulated by the lungs. Both have a powerful impact on acid-base regulation and are regulated by different organ systems. Carbon Dioxide Serum, as bicarbonate acts as one of the reserve alkaline elements in the blood. Bicarbonate neutralizes metabolic acids, such as hydrochloric and lactic acids. We can look at serum CO₂ to help evaluate a trend towards an alkalosis or acidosis in the body. Elevated levels of serum CO₂, or bicarbonate, may bee associated with a trend towards Metabolic Alkalosis and decreased levels are associated with a trend towards Metabolic Acidosis, in this situation, the bicarbonate is being used-up to buffer the increasing levels of acidity or H+ in the body. Serum total carbon dioxide may also be a prognostic factor for 28-day mortality in patients with sepsis.

Uric Acid: In the context of the present invention, the term “Uric acid” refers to a heterocyclic compound of carbon, nitrogen, oxygen, and hydrogen with the formula C₅H₄N₄O₃. It forms ions and salts known as urates and acid urates, such as ammonium acid urate. Uric acid is a product of the metabolic breakdown of purine nucleotides, and it is a normal component of urine. High blood concentrations of uric acid can lead to gout and are associated with other medical conditions, including diabetes and the formation of ammonium acid urate kidney stones. Uric acid (UA) recently emerged as an inflammatory factor that increases oxidative stress and promotes activation of the renin angiotensin aldosterone system. As a consequence, higher UA levels are associated with various stages of the onset and progression of diabetic nephropathy, including metabolic, cardiovascular and kidney function abnormalities. Serum UA levels may serve as a biomarker of renal and cardiovascular risk and as a potential additional therapeutic target in diabetes.

Blood parameter: The value of a “blood parameter” is measurable. By way of example, the blood parameter Na (electrolyte) may have the value 140 mmol ionic sodium per liter blood. The value of a blood parameter may depend on multiple causal variables such as age and breed.

Breed: In the context of the present invention, the term “breed” refers to a stock of animals within a species having a distinctive appearance and typically having been developed by deliberate selection. Thus, the animals are presumably related by descent from common ancestors. By way of example, broiler chicken from the breeds Ross 708 and Cobb 500 can be commercially sourced from local commercial hatcheries.

Carrier: In the context of the present invention, the term “carrier” refers to a sample collection card. Said sample collection card may be of various substrates, such as cardboard, wood, glass, plastic. Preferably, the sample collection card is a filter paper with collection circles printed on their surface, wherein a drop of blood and/or egg yolk may be placed for further analysis. Each collection circle may be used to place one drop of sample. More than one collection circle, allows for placement of more than one sample and separate analysis of the samples.

More preferably, the sample collection card is a chemically treated filter paper designed for the collection, preservation and shipment of dried biological samples for subsequent DNA and RNA analysis, wherein special chemicals lyse and inactivate bacteria and/or viruses and preserve their DNA and RNA for detection by analysis methods, such as PCR. Such sample collection cards are commercially available as Whatman® 903 (GE Healthcare, Piscataway, NJ, EUA) and disclosed in WO2000062023A1. Samples placed on such a carrier and dried are conserved and therefore may be taken virtually anywhere. Dried samples on a carrier do not need refrigerated transportation.

Calcidiol: The terms “25-OH D3”, “25-hydroxy vitamin D3”, “HyD” and “calcidiol” are used interchangeably.

Cholecalciferol: The terms “Cholecalciferol” and “vitamin D₃” are used interchangeably.

Dietary Guidelines: The terms “dietary guidelines”, “feed-based dietary guidelines” or “nutritional guidelines” are used interchangeably and refer to guidelines that are needed to ensure recommended animal nutrient levels and/or improve health and performance factors and are intended to establish a basis for producing nutritionally complete feed rations.

Group of animals: The term “group of animals” refers to preferably at least 10, more preferably to at least 100 and most preferably to at least 1000 animals that are raised in the same compartment (e.g. in the same pen or in the same house).

Health and performance factors: In the context of the present invention, the term “health and performance factors” refers to objective, quantifiable characteristics of animal performance and health. Preferably health and performance factors are selected from the group of, but not limited to, bone strength, meat yield and eggshell strength, milk fever likelihood, hatchability, embryo livability, gut health (Microflora), nutrient absorption, Skeletal Health, Electrolyte Balance, Liver health, Oxidative Stress & Inflammation.

By correlating biomarker levels in the blood and/or yolk with performance improvements it is possible to estimate the effect on economically important parameters. For example, comparing the performance increase in various broiler trials it could be concluded that on average, a 1% increase in the plasma levels of 25-OH-D3 resulted in improvements of 0.029%, 0.173%, 0.008% and 0.296% in ADG, bone strength, breast meat yield and gait score respectively. (Sakas et al., 2019; Bray et al., 2012; Sauders et al 2004; Vignale et al., 2015).

Bone strength: Bone strength is determined by bone geometry, cortical thickness and porosity, trabecular bone morphology, and intrinsic properties of bony tissue. Bone strength may be indirectly estimated by bone mineral density (BMD) using analysis methods, such as dual-energy X-ray absorptiometry (DXA). Osteoporosis is a disease defined by decreased bone mass and alteration of microarchitecture which results in increased bone fragility and increased risk of fracture. The major complication of osteoporosis, i.e., fracture, is due to a lower bone strength. Thus, any treatment of osteoporosis implies an improvement in bone strength.

Meat yield: In the context of the present invention, the term “meat yield” or “lean meat yield” (LMY %) refers to the proportion of a carcase that is lean meat (muscle), expressed as a percentage. The LMY % of a carcase is a standard way to assess the composition of a carcase and does not change depending on the cutting specifications used to market the carcase. LMY % is calculated differently for sheep and beef. For sheep, LMY % is predicted using hot standard carcase weight (HSCW) and knife GR tissue depth. Hot carcass weight is the weight of a carcass prior to chilling. A beef carcass consists of 70 to 75 percent water. As the carcass chills and ages, water will be lost through evaporation. In just the first 24 hours a carcass can lose up to 2 to 5 percent of its initial weight. The higher the HSCW and the lower the GR value the higher the LMY %. The algorithm for beef LMY % is calculated using HSCW, rib fat depth and, in some cases, eye muscle area (EMA). The higher the HSCW and the lower the rib fat depth (and where EMA is used, the larger the EMA), the higher the LMY %. Processors may use different systems to determine LMY % but these systems have all been calibrated against a CT scan—the gold standard system to measure LMY %. This allows processors and producers to compare carcases in a standard way. Producers can manage LMY % through key on-farm practices such as nutrition and genetic selection. Providing information about LMY % on LDL means, producers can make better informed management decisions to maximise carcase value in the future.

Eggshell strength: Shell breakage is and always has been a financial drain on the poultry industry. Methods of measuring eggshell strength may include quasi-static compression. In this destructive process, eggs are compressed between two parallel plates under a steady increasing load until failure results. Force and deformation are continuously recorded and the strength of the eggshell is given in terms of force at failure. Non-destructive deformation of the shell is generally assumed to give a measure of its stiffness characteristics.

Milk fever: In the context of the present invention, the term “Milk fever”, or “postparturient hypocalcemia”, or “parturient paresis” refers to a disease, primarily in dairy cattle but also seen in beef cattle and non-bovine domesticated animals, characterized by reduced blood calcium levels (hypocalcemia). It occurs following parturition, at onset of lactation, when demand for calcium for colostrum and milk production exceeds the body's ability to mobilize calcium. “Fever” is a misnomer, as body temperature during the disease is generally not elevated. Milk fever is more commonly seen in older animals (which have reduced ability to mobilize calcium from bone) and in certain breeds (such as Channel Island breeds).

Hatchability: The term “hatchability” as used herein refers to the percentage of total eggs that hatch a viable chick or poult (e.g., number of poults hatched per number of eggs set×100). Increasing hatchability, that is, increasing the percentage of laid eggs which hatch, is particularly desirable, as even a small increase in percentage will significantly affect the resulting chick numbers.

Therefore, increased hatchability is considered an important factor in large scale breeding programs. An increase in the likelihood an egg will hatch can be measured by calculating the “hatchability” or the “hatch of fertile” of a number of eggs. The term “hatch of fertile” as used herein refers to the percentage of total fertile eggs that hatch a viable chick or poult (e.g., number of poults hatched per number of fertile eggs set×100).

Embryo livability: The term “embryo livability” as used herein refers to the survival expectancy or viability of embryos.

Gut health: The term “embryo livability” as used herein refers to Improving the Diversity of the Microbiome and/or increase the amount of beneficial bacteria of the intestine, and specifically in the colon. The beneficial bacteria which are known to inhabit the colon include Acidaminococcus, Akkermansia sp. Bacteroides ovatus, Bifidobacterium spp., Blautia producta, Clostridium cocleatum, Collinsella aerofaciens, Dorea longicatena, Escherichia coli, Eubacterium spp., Faecalibacterium prausnitzii, Lachnospira pectinoshiza, Lactobacillus spp., Parabacteroides distasonis, Raoultella spp., Roseburia spp., Ruminococcus spp., and Streptococcus spp. Preferably the bacteria which are increased are selected from the group consisting of Bifidobacterium, Akkermansia, Faecalibacterium and Bacteroides. More preferably Bifidobacterium adolescentis, Bifidobacterium longum, Bacteroides ovatus, Bacteroides xylanisolvens, Lachnoclostridium sp. Akkermansia muciniphila, Blautia wexlerae, and/or Faecalibacterium prausnitzii are increased after administration of the antioxidants of this invention. Increasing the diversity of bacteria and/or increasing the amount of beneficial bacteria is particularly helpful when the animal is experiencing a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and cardiovascular disease.

Skeletal Health: The term “skeletal health” as used herein may refer to, but not be limited to, various aspects of skeletal health, such as bone strength, good bone mineralization, a high bone density and/or fracture resistance.

Electrolyte Balance: The term “electrolyte balance” as used herein refers to the absence of an Electrolyte imbalance, or water-electrolyte imbalance, which is an abnormality in the concentration of electrolytes in the body. Electrolytes play a vital role in maintaining homeostasis in the body. They help to regulate heart and neurological function, fluid balance, oxygen delivery, acid-base balance and much more. Electrolyte imbalances can develop by consuming too little or too much electrolyte as well as excreting too little or too much electrolyte. Electrolyte disturbances are involved in many disease processes, and are an important part of patient management in medicine. The causes, severity, treatment, and outcomes of these disturbances can differ greatly depending on the implicated electrolyte.^{[3]} The most serious electrolyte disturbances involve abnormalities in the levels of sodium, potassium or calcium. Other electrolyte imbalances are less common and often occur in conjunction with major electrolyte changes. The kidney is the most important organ in maintaining appropriate fluid and electrolyte balance, but other factors such as hormonal changes and physiological stress play a role.^{[2]}

Liver health: The term “liver health” as used herein refers to maintaining healthy liver functions.

Oxidative Stress: Oxidative stress reflects an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by the reactive oxygen species (ROS) generated, e.g., O₂⁻ (superoxide radical), OH (hydroxyl radical) and H₂O₂ (hydrogen peroxide). Further, some reactive oxidative species act as cellular messengers in redox signaling. Thus, oxidative stress can cause disruptions in normal mechanisms of cellular signaling.

Inflammation: Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair.

The five cardinal signs are heat, pain, redness, swelling, and loss of function (Latin calor, dolor, rubor, tumor, and functio laesa). Inflammation is a generic response, and therefore it is considered as a mechanism of innate immunity, as compared to adaptive immunity, which is specific for each pathogen. Too little inflammation could lead to progressive tissue destruction by the harmful stimulus (e.g. bacteria) and compromise the survival of the organism. In contrast, too much inflammation, in the form of chronic inflammation, is associated with various diseases, such as hay fever, periodontal disease, atherosclerosis, and osteoarthritis.

Inflammation can be classified as either acute or chronic. Acute inflammation is the initial response of the body to harmful stimuli, and is achieved by the increased movement of plasma and leukocytes (in particular granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

High Efficiency Liquid Chromatography Coupled with Mass Spectrometry: High Efficiency Liquid Chromatography Coupled with Mass Spectrometry (LC-MS/MS) is a highly sensitive analysis technique. It reads the analyte, analyte, mass/load (m/z) and its fragments (product ion). It is highly sensitive technique, determining parts per billion (ppb or ng/ml), while only needing small sample volumes of 150 μL (microliters) and suffering only low interference of biological matrix (plasma) and metabolite components.

Nutrient parameter: A or nutrient parameter, for the purpose of this invention, is a substance used by an organism to survive, grow, and reproduce. Nutrients may be classified to describe nutrient needs of animals and divided into macronutrients and micronutrients. Macronutrients, such as carbohydrates, fats, proteins, water, are consumed in relatively large amounts (grams or ounces) and are primarily used to generate energy or to incorporate into tissues for growth and repair. Micronutrients, such as vitamins and dietary minerals, are needed in smaller amounts (milligrams or micrograms). Micronutrients for the purpose of this invention may be, but not limited to, minerals and vitamins. The nutrient parameter may be selected from the group consisting of fat-soluble vitamins, water soluble vitamins, trace minerals. Preferably, the nutrient parameter is selected from Vitamin D₃, 25-OH-D3, Vitamin A and/or Vitamin E.

Raising animals: In the context of the present invention, “raising animals” refers to the production of animals, regardless of the purpose. Thus, “raising animals” includes raising animals for meat and/or egg production. Chicken that are bred for meat production are broiler chicken.

Vitamin D₃: Vitamin D₃ is a fat-soluble vitamin, that needs to go through an enzymatic digestive process (lipase) and micelles formation in the intestine to be absorbed by the enterocytes (Combs Jr. and McClung, 2017). When going through the liver, it is hydroxylated and a first metabolite is produced: 25-OH-D3, which is the most abundant form in the organism and the one that will be available to be metabolized and transformed mainly in the kidney in the active form of vitamin D.

Due to the advantages over dietary Vitamin D₃, the 25-OH-D3 feed inclusion is a strategy currently used in animal nutrition. At the same level of inclusion, the absorption of 25-OH-D3 is higher (74.9%) than vitamin D₃ (66.5%) (Bar et al., 1980). When fed, 25OHD3 doesn't need to go through the transformation stage in the liver (Soares et al., 1995), in comparison to Vitamin D₃ has a more efficient absorption in the intestine, a greater retention, and less excretion (Bar et al., 1980), a biological activity of 2.0 to 2.5 times greater (Soares et al., 1978; Fritts and Waldroup, 2003) and doesn't show toxic effects up to 10 times its recommended dose (Yarger et al., 1995).

Being the 25-OH-D3 the most abundant vitamin D form in the bloodstream, its evaluation is the metric to evaluate vitamin D status in all animal species, including humans. The evaluation of this metabolite has been used to determine the ideal dietary level and make correlations with important parameters such as: calcium and phosphorus plasma levels, bone characteristics (Tizziani et al., 2019), muscle protein synthesis (Vignale et al., 2015; Prokoski et al., 2019), with performance variables (Zhang et al., 2020), and stablish differences between production conditions (DSM Field Trial. Lozano, 2021).

The role of vitamin D in the Calcium and Phosphorus metabolism, the physiological processes and the requirements for poultry are well documented (Rama-Rao et al., 2006; Rama-Rao et al., 2009). Additionally, its role in the regulation of the immune response (Chou et al., 2009; Morris et al., 2014), muscle formation and meat yield (Hutton et al., 2014; Prokoski et al., 2019) are widely described in recent literature.

The optimal level of inclusion of vitamin D₃ has been a subject of research for many decades. The first recommendations were based on studies conducted using purified or semi-purified diets under controlled experimental conditions (NRC, 1994). However, those recommendations have resulted lower than those required under commercial production conditions or under pathogen challenges that generally increase nutrient requirements.

At commercial level, the Vitamin D₃ inclusion varies between 3,000 or 5,000 Ul/kg of feed (Bozkurt et al., 2017; Sakkas et al., 2019). Normally, poultry companies determine the level to be used based on those available recommendations, their own production conditions, expected performance, final product, and maximum return of investment.

In general, measuring 25OHD3 in plasma allow us to know the Vitamin D Status in an animal. There is a correlation between health status, immunity and performance with the Vitamin D3 status. The plasma/blood level of 25OHD3 reached by the animal will be always greater when HyD is supplemented in the feed due to its metabolic advantages over Vitamin D and competitors.

### FIGURES

FIG. 1. Effect of inclusion of dietary 25(OH)D3 (Hy-D®) on blood concentration of vitamin D3 of 21-d-old broiler chickens.

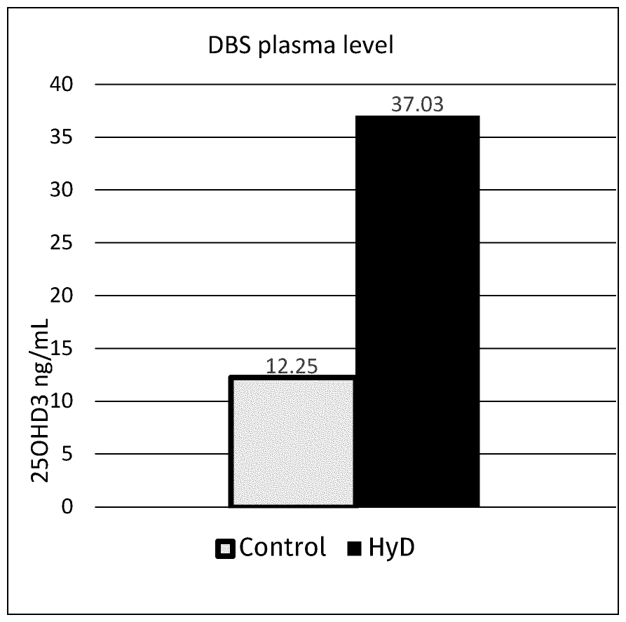

### EMBODIMENTS OF THE INVENTION

1. Method of determining at least one analyte in the blood of an animal, said method comprising the steps:
vii) providing a drop of blood from an animal,
viii) collecting the blood provided in step i) on a carrier,
ix) letting the blood on the carrier dry, and
x) analyzing the blood on the carrier provided in step iii) to measure the values of at least one analyte.

2. Method according to claim 1, further comprising the steps of
xi) Comparing the values of the at least one analyte measured in step iv) with the levels of the at least one analyte recommended in nutritional guidelines, and
xii) adapting feed that is fed to the animal, if the values of the at least one analyte measured in step iv) deviate from ranges recommended in the nutritional guidelines, to ensure recommended animal nutrient levels according to the nutritional guidelines.

3. Method of raising a group of animals of same species and same breed, said method comprising the steps:
i) providing a drop of blood from at least one member of the group,
ii) collecting the blood provided in step i) on a carrier,
iii) letting the blood on the carrier dry,
iv) analyzing the blood on the carrier provided in step iii) to measure the values of at least one analyte, and
v) Comparing the values of the at least one analyte measured in step iv) with the levels of the at least one analyte recommended in nutritional guidelines
vi) adapting feed that is fed to the animal, if the values of the at least one analyte measured in step iv) deviate from ranges recommended in the nutritional guidelines, to ensure recommended animal nutrient levels according to the nutritional guidelines.

4. Method according to any of claims 1 to 3, wherein the blood is venous blood.

5. Method of determining at least one analyte in egg yolk, said method comprising the steps:
vii) providing a drop of egg yolk,
viii) collecting the egg yolk provided in step i) on a carrier,
ix) letting the egg yolk on the carrier dry, and
x) analyzing the egg yolk on the carrier provided in step iii) to measure the values of at least one analyte in egg yolk.

6. Method according to claim 5, further comprising the steps of
xi) Comparing the values of the at least one analyte measured in step iv) with the levels of the at least one analyte recommended in nutritional guidelines, and
xii) adapting feed that is fed to the animal, if the values of the at least one analyte measured in step iv) deviate from ranges recommended in the nutritional guidelines, to ensure recommended animal nutrient levels according to the nutritional guidelines.

7. Method of raising a group of animals of same species and same breed, said method comprising the steps:
vii) providing a drop of egg yolk from an egg of least one member of the group,
viii) collecting the egg yolk provided in step i) on a carrier,
ix) letting the egg yolk on the carrier dry,
x) analyzing the egg yolk on the carrier provided in step iii) to measure the values of at least one analyte, and
xi) Comparing the values of the at least one analyte measured in step iv) with the levels of the at least one analyte recommended in nutritional guidelines
xii) adapting feed that is fed to the animal, if the values of the at least one analyte measured in step iv) deviate from ranges recommended in the nutritional guidelines, to ensure recommended animal nutrient levels according to the nutritional guidelines.

8. Method according to any of claims 1 to 7, wherein the carrier is a sample collection card.

9. Method according to claim 8, wherein the sample collection card is carton covered with a substrate for lysing cells and purifying nucleic acid.

10. Method according to claim 9, wherein the sample collection card is a Whatman® 903 (GE Healthcare, Piscataway, NJ, EUA).

11. Method according to any of claims 1 to 10, wherein the analysis in step iv) is performed by liquid chromatography coupled with mass spectrometry (LC-MS/MS).

12. Method according to any of claims 1 to 11, wherein the analyte is a nutrient.

13. Method according to any of claims 1 to 12, wherein the analyte is selected from the group consisting of fat-soluble vitamins, water soluble vitamins, trace minerals.

14. Method according to any of claims 1 to 13, wherein the analyte is selected from the 25-OH-D3, Vitamin A and/or Vitamin E.

15. Method according to any of claims 1 to 11, wherein the analyte is a biomarker.

16. Method according to any of claim 1 to 11 or 15, wherein the analyte is selected from the group consisting of Albumin, Calcium, Carotenoids, Creatine Kinease, Globulin, Glucose, Hemoglobin, Phosphorus, Potassium, Sodium, Total Carbon Dioxide, Uric Acid.

17. Method according to any of claim 1 to 13 or 15 or 16, wherein the health and performance factors is selected from the group of bone strength, meat yield and eggshell strength, milk fever likelihood, hatchability, embryo livability, gut health, nutrient absorption, Skeletal Health, Electrolyte Balance, Liver health, Oxidative Stress & Inflammation.

18. Method according to any of claims 1 to 17, wherein the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

19. Method according to any of claims 1 to 18, wherein the animal is selected from the group of pigs or swine (including, but not limited to, piglets, growing pigs, and sows) or poultry (including but not limited to poultry, turkey, duck, quail, guinea fowl, goose, pigeon, squab, chicken, broiler, layer, pullet and chick).

### EXAMPLES

### Example 1 Poultry Sampling

### 1. Birds and Housing

560 one-d-old broiler chickens were used from 1 to 21 d-old in the Metabolism Room facility of the Laboratory of Studies and Research on Production and Nutrition of Non-Ruminant Animals (LEPNAN), Federal University of Paraná, Curitiba, Brazil. The birds were housed in metabolic cages with 4 floors, with 2 cages per floor, measuring 0.98×0.90×0.50 m (length×width×height). All cages were equipped with trough feeders and nipple drinkers. Max/min thermometers were used for temperature verification.

Birds received feed and water ad libitum. The lighting programs, as well as the adequate room temperature, was set according to the breeders guidelines, with the respective use of digital timers and electrical portable heaters. The room will have natural ventilation, controlled by the opening and closing of windows. The daily routine will include the verification of temperature, feed offering, and inspection of cages for dead birds, which will be removed and their weight will be noted for calculation of mortality rate.

### 2. Experimental Design

A completely randomized design will be carried out, with or without Hy-D® (Table 1), totaling 2 treatments with 7 replicates of 40 birds each.

TABLE 1
Experimental treatments.

| Treatment | Hy-D ® |
|---|---|
| T1 | 0 |
| T2 | 250 g/ton* |

*g of the premix Hy-D ®/ton of feed, providing 69 mg of 25-OH-D₃/ton of feed.

### 3. Experimental Diets' Preparation

Diets will be offered in mash form and based on corn and soybean meal, formulated to attend the nutritional requirements of starter phase broilers. Diets will vary according to the inclusion of Hy-D® (With or without 250 g/ton of feed).

### 4. Analyzed Variables

At 20 days, blood samples were collected from 75 randomly chosen birds per group (control, HyD) for analysis of levels of vitamin D metabolites, measured via Dried Blood Spots (DBS) method.

5.

TABLE 3
Experimental diets' composition

| | Without inclusion | With inclusion |
|---|---|---|
| Ingredients (%) | of vitamin D | of vitamin D |
| Corn | 54.86 | 54.86 |
| Soybean meal | 38.60 | 38.60 |
| Soybean oil | 2.30 | 2.30 |
| Limestone | 1.01 | 1.01 |
| Dicalcium phosphate | 1.00 | 1.00 |
| Sodium chloride | 0.49 | 0.49 |
| L-Lysine HCl | 0.118 | 0.118 |
| L-Threonine | 0.06 | 0.06 |
| DL-Methionine | 0.276 | 0.276 |
| Choline Chloride | 0.10 | 0.10 |
| Vitamin Premix¹ | 0.10 | 0.10 |
| Mineral Premix² | 0.05 | 0.05 |
| Phytase³ | 0.0075 | 0.0075 |
| Vitamin D⁴ | 0.00 | 0.025 |
| Celite⁵ (Marker) | 1.025 | 1.00 |
| Metabolizable | 3,048 | 3,048 |
| energy (kcal) | | |
| Sodium (%) | 0.21 | 0.21 |
| Chlorine (%) | 0.36 | 0.36 |
| Digestible | 1.22 | 1.22 |
| lysine (%) | | |
| Digestible | 0.58 | 0.58 |
| methionine (%) | | |
| Methionine + | 0.90 | 0.90 |
| cysteine (%) | | |
| Digestible | 0.82 | 0.82 |
| threonine (%) | | |
| Crude protein (%) | 23.32 | 23.33 |
| Calcium (%) | 0.92 | 0.86 |
| Total phosphorus (%) | 0.59 | 0.58 |
| Ash (%) | 7.13 | 7.54 |

¹Supplied per kilogram of product: copper 20 g; iron, 100 g; iodine, 2 g; manganese, 130 g; zinc, 130 g.
²Supplied per kilogram of product: vitamin A, 11,000,000 UI; vitamin D3, 4,000,000 UI; vitamin E, 55,000 UI; vitamin K3, 3 g; vitamin B1, 2.3 g; vitamin B2, 7 g; pantothenic acid, 12 g; vitamin B6, 4 g; vitamin B12, 25 mg; nicotinic acid, 60 g; folic acid, 2 g; biotin, 250 mg; selenium, 300 mg.
³RONOZYME ® HiPhos GT with 20,000 FYT/g (DSM Nutritional Products - Kaiseraugst, Switzerland)
⁴25(OH)D₃ (Hy-D ®, DSM Nutritional Products - Kaiseraugst, Switzerland).
⁵Celite ® Insoluble marker (Celite ® 400 - Celite Corp., Lompoc, US).

### 6. Dried Blood Spots Analysis

TABLE 4
Effect of inclusion of dietary 25(OH)D3 (Hy-D ®) on blood
concentration of vitamin D3 of 21-d-old broiler chickens.

| | Control | HyD |
|---|---|---|
| Average | 12.25 | 37.03 |
| Std Dev | 3.44 | 7.25 |
| n | 75 | 75 |

### Example 2 Blood Sampling Instructions

1A (Poultry)—Hold the bird and puncture the wing vein or take the sample with a syringe
1B (Swine)—Restrain the pig and puncture a marginal ear vein located on the ear dorsal surface and take the sample with a syringe
2-Carefully collect a drop of blood using the FTA card. Touch the blood with the FTA and put the sample in each circle.
3-Repeat the process in the other field (circle) with another animal
4-Allow the sample to dry until the coloration adopts a brown (dry blood) characteristic.
5-Identify the card correctly with animal data. E. g. Identify each sample with the corresponding project (customer) and animal information: age, sex, day of collection, farm, and treatment.
6-Put the samples in a plastic bag, removing as much air as possible to avoid samples not suitable for analysis.

The sampling does not require any form of processing, such as drying or homogenization, prior to collection of the sample using the FTA card.

### Example 3 Egg Yolk Sampling Instructions

1—Break the egg, take a yolk sample with a syringe or pipette
2—Carefully collect a drop of yolk using the FTA card. Touch the yolk with the FTA and put the sample in each circle. One circle per sample
3—Repeat the process in the other field (circle) with another sample
4—Allow the sample to dry
5—Identify the card correctly with animal data. E. g. Identify each sample with the corresponding project (customer) and animal information: age, sex, day of collection, farm, and treatment.
6—Put the samples in a plastic bag, removing as much air as possible to avoid samples not suitable for analysis.

The sampling does not require any form of processing, such as drying or homogenization, prior to collection of the sample using the FTA card.

1. Method of determining at least one analyte in the blood of an animal, said method comprising the steps:
i) providing a drop of blood from an animal,
ii) collecting the blood provided in step i) on a carrier,
iii) letting the blood on the carrier dry, and
iv) analyzing the blood on the carrier provided in step iii) to measure the values of at least one analyte.

2. Method according to claim 1, further comprising the steps of
v) Comparing the values of the at least one analyte measured in step iv) with the levels of the at least one analyte recommended in nutritional guidelines, and
vi) adapting feed that is fed to the animal, if the values of the at least one analyte measured in step iv) deviate from ranges recommended in the nutritional guidelines, to ensure recommended animal nutrient levels according to the nutritional guidelines.

3. Method of raising a group of animals of same species and same breed, said method comprising the steps:
i) providing a drop of blood from at least one member of the group,
ii) collecting the blood provided in step i) on a carrier,
iii) letting the blood on the carrier dry,
iv) analyzing the blood on the carrier provided in step iii) to measure the values of at least one analyte, and
v) comparing the values of the at least one analyte measured in step iv) with the levels of the at least one analyte recommended in nutritional guidelines
vi) adapting feed that is fed to the animal, if the values of the at least one analyte measured in step iv) deviate from ranges recommended in the nutritional guidelines, to ensure recommended animal nutrient levels according to the nutritional guidelines.

4. Method according to claim 1, wherein the blood is venous blood.

5. Method of determining at least one analyte in egg yolk, said method comprising the steps:
i) providing a drop of egg yolk,
ii) collecting the egg yolk provided in step i) on a carrier,
iii) letting the egg yolk on the carrier dry, and
iv) analyzing the egg yolk on the carrier provided in step iii) to measure the values of at least one analyte in egg yolk.

6. Method according to claim 5, further comprising the steps of
v) comparing the values of the at least one analyte measured in step iv) with the levels of the at least one analyte recommended in nutritional guidelines, and
vi) adapting feed that is fed to the animal, if the values of the at least one analyte measured in step iv) deviate from ranges recommended in the nutritional guidelines, to ensure recommended animal nutrient levels according to the nutritional guidelines.

7. Method of raising a group of animals of same species and same breed, said method comprising the steps:
i) providing a drop of egg yolk from an egg of least one member of the group,
ii) collecting the egg yolk provided in step i) on a carrier,
iii) letting the egg yolk on the carrier dry,
iv) analyzing the egg yolk on the carrier provided in step iii) to measure the values of at least one analyte, and
v) Comparing the values of the at least one analyte measured in step iv) with the levels of the at least one analyte recommended in nutritional guidelines
vi) adapting feed that is fed to the animal, if the values of the at least one analyte measured in step iv) deviate from ranges recommended in the nutritional guidelines, to ensure recommended animal nutrient levels according to the nutritional guidelines.

8. Method according to claim 1, wherein the carrier is a sample collection card.

9. Method according to claim 8, wherein the sample collection card is carton covered with a substrate for lysing cells and purifying nucleic acid.

10. Method according to claim 9, wherein the sample collection card is a Whatman® 903 (GE Healthcare, Piscataway, NJ, EUA).

11. Method according to claim 1, wherein the analysis in step iv) is performed by liquid chromatography coupled with mass spectrometry (LC-MS/MS).

12. Method according to claim 1, wherein the analyte is a nutrient.

13. Method according to claim 1, wherein the analyte is selected from the group consisting of fat-soluble vitamins, water soluble vitamins, trace minerals.

14. Method according to claim 1, wherein the analyte is selected from the 25-OH-D₃, Vitamin A and/or Vitamin E.

15. Method according to claim 1, wherein the analyte is a biomarker.

16. Method according to claim 1, wherein the analyte is selected from the group consisting of Albumin, Calcium, Carotenoids, Creatine Kinease, Globulin, Glucose, Hemoglobin, Phosphorus, Potassium, Sodium, Total Carbon Dioxide, Uric Acid.

17. Method according to claim 1, wherein the health and performance factors is selected from the group of bone strength, meat yield and eggshell strength, milk fever likelihood, hatchability, embryo livability, gut health, nutrient absorption, Skeletal Health, Electrolyte Balance, Liver health, Oxidative Stress & Inflammation.

18. Method according to claim 1, wherein the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

19. Method according to claim 1, wherein the animal is selected from the group of pigs or swine (including, but not limited to, piglets, growing pigs, and sows) or poultry (including but not limited to poultry, turkey, duck, quail, guinea fowl, goose, pigeon, squab, chicken, broiler, layer, pullet and chick).
